(19) United States
(12) Patent Application Publication
(10) Pub. No.: US 2025/0025223 A1
(43) Pub. Date: Jan. 23, 2025

(54) ELECTRICAL ISOLATION OF ELECTROSURGICAL INSTRUMENT END EFFECTORS

(71) Applicant: CILAG GMBH INTERNATIONAL, Zug (CH)

(72) Inventors: Niko MURRELL, Blue Ash, OH (US); Joseph NATH, Cincinnati, OH (US); Kevin MONTGOMERY, Cincinnati, OH (US); Mark R. BURCHNALL, Loveland, OH (US); Catherine M. PASSMORE, Lawrenceville, GA (US); Robert G. STATES, III, Morrow, OH (US)

(73) Assignee: CILAG GMBH INTERNATIONAL, Zug (CH)

(21) Appl. No.: 18/590,168

(22) Filed: Feb. 28, 2024

Related U.S. Application Data
(60) Provisional application No. 63/514,731, filed on Jul. 20, 2023.

(51) Int. Cl. A61B 18/14; A61B 17/29; A61B 18/00; A61B 34/00; A61B 34/37

(52) U.S. Cl. CPC A61B 18/1445; A61B 34/37; A61B 34/71; A61B 2017/2932; A61B 2018/00083; A61B 2018/00178

(57) ABSTRACT

An end effector for a surgical tool includes a jaw including a shank and a contact plate forming part of the shank, and a jaw holder secured to the jaw and providing a first molded component defining a cavity and a cable passage that communicates with the contact plate, a cable clip received within the cavity, and a second molded component overmolded onto the first molded component and at least a portion of the cable clip, and filling the cavity. An electrical conductor extends through the cable passage and includes an insulation sheath and a supply conductor electrically coupled to the contact plate to supply electrical energy to the jaw. The cable clip covers an exposed portion of the insulation sheath within the cavity, and the second molded component encapsulates and electrically insulates the contact plate and an exposed portion of the supply conductor.

### CROSS-REFERENCE TO RELATED APPLICATIONS

The present application is a non-provisional patent application claiming priority to U.S. Provisional Patent Application Ser. No. 63/514,731, entitled “Electrical Isolation of Electrosurgical Instrument End Effectors,” and filed on Jul. 20, 2023, the contents of which are hereby incorporated by reference in their entirety.

### BACKGROUND

Minimally invasive surgical (MIS) instruments are often preferred over traditional open surgical devices due to reduced post-operative recovery time and minimal scarring. Laparoscopic surgery is one type of MIS procedure in which one or more small incisions are formed in the abdomen of a patient and a trocar is inserted through the incision to form a pathway that provides access to the abdominal cavity. Through the trocar, a variety of instruments and surgical tools can be introduced into the abdominal cavity. The trocar also helps facilitate insufflation to elevate the abdominal wall above the organs. The instruments and tools introduced into the abdominal cavity via the trocar can be used to engage and/or treat tissue in a number of ways to achieve a diagnostic or therapeutic effect.

Various robotic systems have recently been developed to assist in MIS procedures. Robotic systems can allow for more intuitive hand movements by maintaining natural eye-hand axis. Robotic systems can also allow for more degrees of freedom in movement by including a “wrist” joint that creates a more natural hand-like articulation. The instrument's end effector can be articulated (moved) using a cable driven motion system having one or more drive cables that extend through the wrist joint. A user (e.g., a surgeon) is able to remotely operate an instrument's end effector by grasping and manipulating in space one or more controllers that communicate with a tool driver coupled to the surgical instrument. User inputs are processed by a computer system incorporated into the robotic surgical system and the tool driver responds by actuating the cable driven motion system and, more particularly, the drive cables. Moving the drive cables articulates the end effector to desired positions and configurations.

Some surgical tools are electrically energized and commonly referred to as electrosurgical instruments. An electrosurgical instrument has a distally mounted end effector that includes one or more electrodes. When supplied with electrical energy, the end effector electrodes are able to generate heat sufficient to cut, cauterize, and/or seal tissue.

Electrosurgical instruments can be configured for bipolar or monopolar operation. In bipolar operation, current is introduced into and returned from the tissue by active and return electrodes, respectively, of the end effector. Electrical current in bipolar operation is not required to travel long distances through the patient before returning to the return electrode. Consequently, the amount of electrical current required is minimal, which greatly reduces the risk of accidental ablations and/or burns. In addition, the two electrodes are closely spaced and within the surgeon's field of view, which further reduces the risk of unintended ablations and burns.

In monopolar operation, current is introduced into the tissue by an active (or source) end effector electrode and returned through a return electrode (e.g., a grounding pad) separately located on a patient's body. Monopolar electrosurgical instruments facilitate several surgical functions, such as cutting tissue, coagulating tissue to stop bleeding, or concurrently cutting and coagulating tissue. The surgeon can apply a current whenever the conductive portion of the instrument is in electrical proximity with the patient, permitting the surgeon to operate with monopolar electrosurgical instruments from many different angles.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an example robotic surgical system that may incorporate some or all of the principles of the present disclosure.

FIG. 2 is an isometric side view of an example surgical tool that may incorporate some or all of the principles of the present disclosure.

FIG. 3 illustrates potential degrees of freedom in which the wrist of the surgical tool of FIG. 2 may be able to articulate (pivot) and translate.

FIG. 4 is an enlarged isometric view of the distal end of the surgical tool of FIG. 2.

FIG. 5 is an enlarged isometric view of a portion of the end effector of FIG. 4, according to one or more embodiments.

FIGS. 6A-6D illustrate progressive views of an example process of manufacturing the portion of the end effector of FIG. 5.

FIGS. 7A-7D illustrate progressive views of an example process of manufacturing the portion of the end effector of FIG. 5, according to one or more embodiments of the present disclosure.

FIGS. 8A-8C illustrate progressive views of another example process of manufacturing the portion of the end effector of FIG. 5, according to one or more additional embodiments of the present disclosure.

FIGS. 9A-9C depict alternate examples of manufacturing the jaw holder of the end effector of FIG. 5, according to one or more additional embodiments.

FIGS. 10A and 10B are isometric and cross-sectional side views of another example method of manufacturing the jaw holder of the end effector of FIG. 5, according to one or more additional embodiments.

### DETAILED DESCRIPTION

The present disclosure is related to robotic surgical systems and, more particularly, to electrosurgical instruments having an end effector designed to insulate an electrical conductor from conductive materials that form part of the end effector.

Embodiments discussed herein describe electrosurgical instruments that use electrical energy to perform a variety of surgical procedures. The end effectors described herein provide jaw holders that have been developed to provide a positive pressure shutoff on an electrical conductor at the cable exit for the jaw holder, without risk of damage to the insulation sheath of the electrical conductor. Other end effector embodiments provide jaw holders that have been developed to reduce or prevent the temperature and pressure of an advancing mold front at the cable exit during an injection molding process.

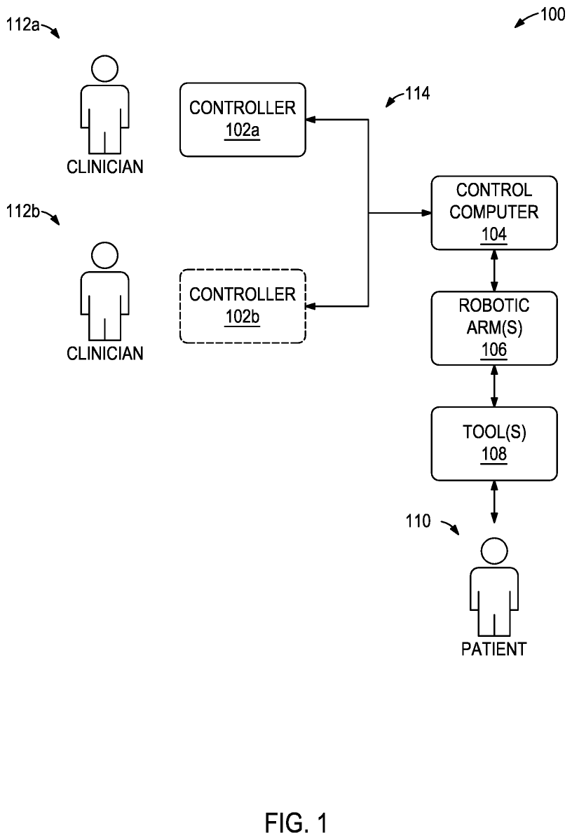

FIG. 1 is a block diagram of an example robotic surgical system 100 that may incorporate some or all of the principles of the present disclosure. As illustrated, the system 100 can include at least one set of user input controllers 102a and at least one control computer 104. The control computer 104 may be mechanically and/or electrically coupled to a robotic manipulator and, more particularly, to one or more robotic arms 106 (alternately referred to as “tool drivers”). In some embodiments, the robotic manipulator may be included in or otherwise mounted to an arm cart capable of making the system portable. Each robotic arm 106 may provide a location for mounting one or more surgical instruments or tools 108 for performing various surgical tasks on a patient 110. Operation of the robotic arms 106 and associated tools 108 may be directed by a clinician 112a (e.g., a surgeon) from the user input controller 102a.

In some embodiments, a second set of user input controllers 102b (shown in dashed line) may be operated by a second clinician 112b to direct operation of the robotic arms 106 and tools 108 via the control computer 104 and in conjunction with the first clinician 112a. In such embodiments, for example, each clinician 112a,b may control different robotic arms 106 or, in some cases, complete control of the robotic arms 106 may be passed between the clinicians 112a,b as needed. In some embodiments, additional robotic manipulators having additional robotic arms may be utilized during surgery on the patient 110, and these additional robotic arms may be controlled by one or more of the user input controllers 102a,b.

The control computer 104 and the user input controllers 102a,b may be in communication with one another via a communications link 114, which may be any type of wired or wireless telecommunications means configured to carry a variety of communication signals (e.g., electrical, optical, infrared, etc.) according to any communications protocol. In some applications, for example, there is a tower with ancillary equipment and processing cores designed to drive the robotic arms 106.

The user input controllers 102a,b generally include one or more physical controllers that can be grasped by the clinicians 112a,b and manipulated in space while the surgeon views the procedure via a stereo display. The physical controllers generally comprise manual input devices movable in multiple degrees of freedom, and which often include an actuatable handle for actuating the surgical tool(s) 108, for example, for opening and closing opposing jaws, applying an electrical potential (current) to an electrode, or the like. The control computer 104 can also include an optional feedback meter viewable by the clinicians 112a,b via a display to provide a visual indication of various surgical instrument metrics, such as the amount of force being applied to the surgical instrument (i.e., a cutting instrument or dynamic clamping member).

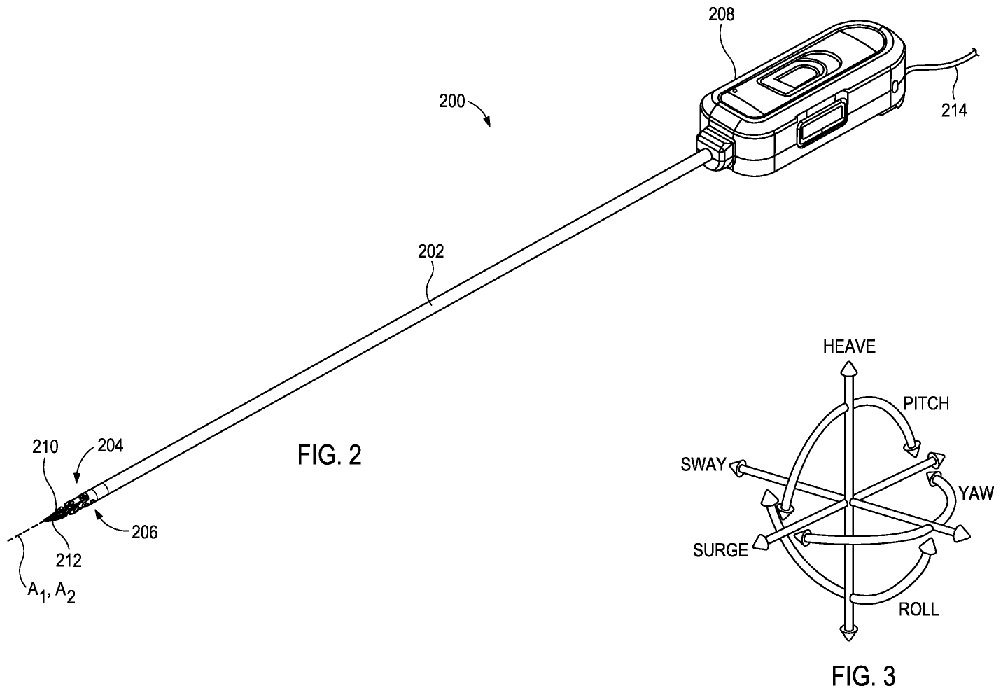

FIG. 2 is an isometric side view of an example surgical tool 200 that may incorporate some or all of the principles of the present disclosure. The surgical tool 200 may be the same as or similar to the surgical tool(s) 108 of FIG. 1 and, therefore, may be used in conjunction with a robotic surgical system, such as the robotic surgical system 100 of FIG. 1. Accordingly, the surgical tool 200 may be designed to be releasably coupled to a tool driver included in the robotic surgical system 100. In other embodiments, however, aspects of the surgical tool 200 may be adapted for use in a manual or hand-operated manner, without departing from the scope of the disclosure.

As illustrated, the surgical tool 200 includes an elongated shaft 202, an end effector 204, a wrist 206 (alternately referred to as a “wrist joint” or an “articulable wrist joint”) that couples the end effector 204 to the distal end of the shaft 202, and a drive housing 208 coupled to the proximal end of the shaft 202. In applications where the surgical tool is used in conjunction with a robotic surgical system (e.g., the robotic surgical system 100 of FIG. 1), the drive housing 208 can include coupling features that releasably couple the surgical tool 200 to the robotic surgical system.

The terms “proximal” and “distal” are defined herein relative to a robotic surgical system having an interface configured to mechanically and electrically couple the surgical tool 200 (e.g., the housing 208) to a robotic manipulator. The term “proximal” refers to the position of an element closer to the robotic manipulator and the term “distal” refers to the position of an element closer to the end effector 204 and thus further away from the robotic manipulator. Alternatively, in manual or hand-operated applications, the terms “proximal” and “distal” are defined herein relative to a user, such as a surgeon or clinician. The term “proximal” refers to the position of an element closer to the user and the term “distal” refers to the position of an element closer to the end effector 204 and thus further away from the user. Moreover, the use of directional terms such as above, below, upper, lower, upward, downward, left, right, and the like are used in relation to the illustrative embodiments as they are depicted in the figures, the upward or upper direction being toward the top of the corresponding figure and the downward or lower direction being toward the bottom of the corresponding figure.

During use of the surgical tool 200, the end effector 204 is configured to move (pivot) relative to the shaft 202 at the wrist 206 to position the end effector 204 at desired orientations and locations relative to a surgical site. To accomplish this, the housing 208 includes (contains) various drive inputs and mechanisms (e.g., gears, actuators, etc.) designed to control operation of various features associated with the end effector 204 (e.g., clamping, firing, cutting, rotation, articulation, etc.). In at least some embodiments, the shaft 202, and hence the end effector 204 coupled thereto, is configured to rotate about a longitudinal axis A1 of the shaft 202. In such embodiments, at least one of the drive inputs included in the housing 208 is configured to control rotational movement of the shaft 202 about the longitudinal axis A1.

The shaft 202 is an elongate member extending distally from the housing 208 and has at least one lumen extending therethrough along its axial length. In some embodiments, the shaft 202 may be fixed to the housing 208, but could alternatively be rotatably mounted to the housing 208 to allow the shaft 202 to rotate about the longitudinal axis A1. In yet other embodiments, the shaft 202 may be releasably coupled to the housing 208, which may allow a single housing 208 to be adaptable to various shafts having different end effectors.

The end effector 204 can exhibit a variety of sizes, shapes, and configurations. In the illustrated embodiment, the end effector 204 comprises a tissue grasper that includes opposing first (upper) and second (lower) jaws 210, 212 configured to move (articulate) between open and closed positions. As will be appreciated, however, the opposing jaws 210, 212 may alternatively form part of other types of end effectors such as, but not limited to, surgical scissors, a vessel sealer, a clip applier, a needle driver, a babcock including a pair of opposed grasping jaws, bipolar jaws (e.g., bipolar Maryland grasper, forceps, a fenestrated grasper, etc.), etc. One or both of the jaws 210, 212 may be configured to pivot to articulate the end effector 204 between the open and closed positions.

FIG. 3 illustrates the potential degrees of freedom in which the wrist 206 may be able to articulate (pivot) and thereby move the end effector 204. The wrist 206 can have any of a variety of configurations. In general, the wrist 206 comprises a joint configured to allow pivoting movement of the end effector 204 relative to the shaft 202. The degrees of freedom of the wrist 206 are represented by three translational variables (i.e., surge, heave, and sway), and by three rotational variables (i.e., Euler angles or roll, pitch, and yaw). The translational and rotational variables describe the position and orientation of the end effector 204 with respect to a given reference Cartesian frame. As depicted in FIG. 3, “surge” refers to forward and backward translational movement, “heave” refers to translational movement up and down, and “sway” refers to translational movement left and right. With regard to the rotational terms, “roll” refers to tilting side to side, “pitch” refers to tilting forward and backward, and “yaw” refers to turning left and right.

The pivoting motion can include pitch movement about a first axis of the wrist 206 (e.g., X-axis), yaw movement about a second axis of the wrist 206 (e.g., Y-axis), and combinations thereof to allow for 360° rotational movement of the end effector 204 about the wrist 206. In other applications, the pivoting motion can be limited to movement in a single plane, e.g., only pitch movement about the first axis of the wrist 206 or only yaw movement about the second axis of the wrist 206, such that the end effector 204 moves only in a single plane.

Referring again to FIG. 2, the surgical tool 200 may also include a plurality of drive cables (obscured in FIG. 2) that form part of a cable driven motion system configured to facilitate actuation and articulation of the end effector 204 relative to the shaft 202. Moving (actuating) one or more of the drive cables moves the end effector 204 between an unarticulated position and an articulated position. The end effector 204 is depicted in FIG. 2 in the unarticulated position where a longitudinal axis A2 of the end effector 204 is substantially aligned with the longitudinal axis A1 of the shaft 202, such that the end effector 204 is at a substantially zero angle relative to the shaft 202. Due to factors such as manufacturing tolerance and precision of measurement devices, the end effector 204 may not be at a precise zero angle relative to the shaft 202 in the unarticulated position, but nevertheless be considered “substantially aligned” thereto. In the articulated position, the longitudinal axes A1, A2 would be angularly offset from each other such that the end effector 204 is at a non-zero angle relative to the shaft 202.

In some embodiments, the surgical tool 200 may be supplied with electrical power (current) via a power cable 214 coupled to the housing 208. In other embodiments, the power cable 214 may be omitted and electrical power may be supplied to the surgical tool 200 via an internal power source, such as one or more batteries, capacitors, or fuel cells. In such embodiments, the surgical tool 200 may alternatively be characterized and otherwise referred to as an “electrosurgical instrument” capable of providing electrical energy to the end effector 204.

The power cable 214 may place the surgical tool 200 in electrical communication with a generator (not shown) that supplies energy, such as electrical energy (e.g., radio frequency energy), ultrasonic energy, microwave energy, heat energy, or any combination thereof, to the surgical tool 200 and, more particularly, to the end effector 204. Accordingly, the generator may comprise a radio frequency (RF) source, an ultrasonic source, a direct current source, and/or any other suitable type of electrical energy source that may be activated independently or simultaneously.

In applications where the surgical tool 200 is configured for bipolar operation, the power cable 214 will include a supply conductor and a return conductor. Current can be supplied from the generator to an active (or source) electrode located at the end effector 204 via the supply conductor, and current can flow back to the generator via a return electrode located at the end effector 204 via the return conductor. In the case of a bipolar grasper with opposing jaws, for example, the jaws serve as the electrodes where the proximal end of the jaws are isolated from one another and the inner surface of the jaws (i.e., the area of the jaws that grasp tissue) apply the current in a controlled path through the tissue. In applications where the surgical tool 200 is configured for monopolar operation, the generator transmits current through a supply conductor to an active electrode located at the end effector 204, and current is returned (dissipated) through a return electrode (e.g., a grounding pad) separately coupled to a patient's body.

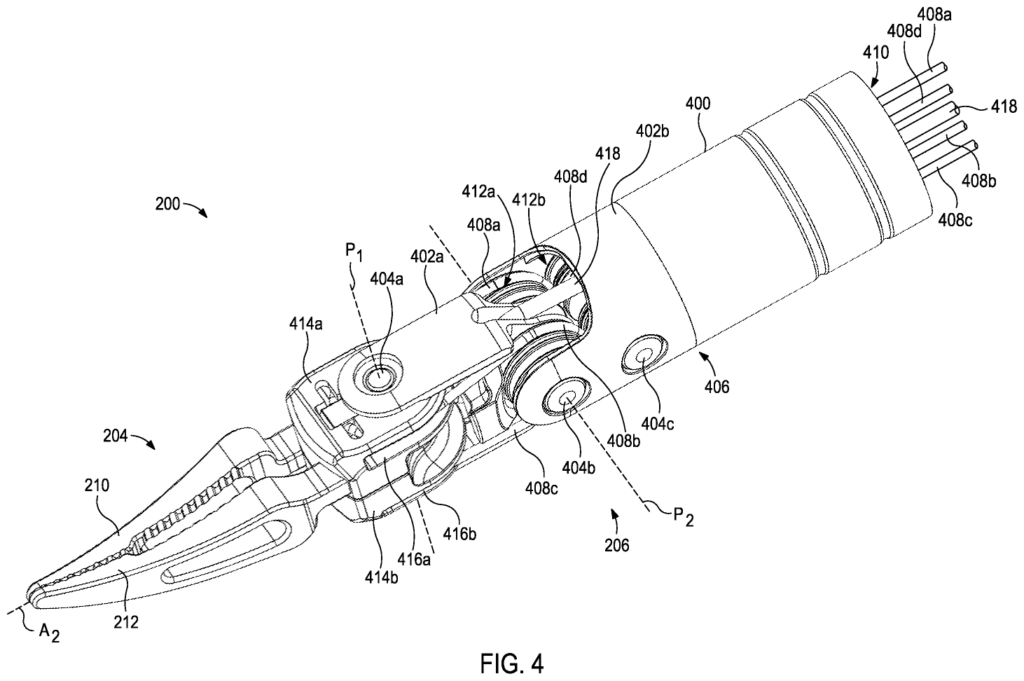

FIG. 4 is an enlarged isometric view of the distal end of the surgical tool 200 of FIG. 2. More specifically, FIG. 4 depicts enlarged views of the end effector 204 and the wrist 206, with the end effector 204 in the unarticulated position. The wrist 206 operatively couples the end effector 204 to the shaft 202 (FIG. 2). In the illustrated embodiment, however, a shaft adapter 400 may be directly coupled to the wrist 206 and otherwise interpose the shaft 202 and the wrist 206. In other embodiments, the shaft adapter 400 may be omitted and the shaft 202 may instead be directly coupled to the wrist 206, without departing from the scope of the disclosure. As used herein, the term “operatively couple” refers to a direct or indirect coupling engagement. Accordingly, the wrist 206 may be operatively coupled to the shaft 202 either through a direct coupling engagement where the wrist 206 is directly coupled to the distal end of the shaft 202, or an indirect coupling engagement where the shaft adapter 400 interposes the wrist 206 and the distal end of the shaft 202.

To operatively couple the end effector 204 to the shaft 202 (e.g., via the shaft adapter 400), the wrist 206 includes a distal clevis 402a and a proximal clevis 402b. The end effector 204 (i.e., the jaws 210, 212) is rotatably mounted to the distal clevis 402a at a first axle 404a, the distal clevis 402a is rotatably mounted to the proximal clevis 402b at a second axle 404b, and the proximal clevis 402b is coupled to a distal end 406 of the shaft adapter 400 (or alternatively the distal end of the shaft 202).

The wrist 206 provides a first pivot axis P₁ that extends through the first axle 404a and a second pivot axis P₂ that extends through the second axle 404b. The first pivot axis P₁ is substantially perpendicular (orthogonal) to the longitudinal axis A₂ of the end effector 204, and the second pivot axis P₂ is substantially perpendicular (orthogonal) to both the longitudinal axis A₂ and the first pivot axis P₁. Movement about the first pivot axis P₁ provides “yaw” articulation of the end effector 204, and movement about the second pivot axis P₂ provides “pitch” articulation of the end effector 204. In the illustrated embodiment, the jaws 210, 212 are mounted at the first pivot axis P₁, thereby allowing the jaws 210, 212 to pivot relative to each other to open and close the end effector 204 or alternatively pivot in tandem to articulate the orientation of the end effector 204.

A plurality of drive cables, shown as drive cables 408a, 408b, 408c, and 408d, extend longitudinally within a lumen 410 defined by the shaft adapter 400 (and/or the shaft 202 of FIG. 2) and pass through the wrist 206 to be operatively coupled to the end effector 204. While four drive cables 408a-d are depicted in FIG. 4, more or less than four drive cables 408a-d may be included, without departing from the scope of the disclosure.

The drive cables 408a-d form part of the cable driven motion system briefly described above, and may be referred to and otherwise characterized as cables, bands, lines, cords, wires, ropes, strings, twisted strings, elongate members, etc. The drive cables 408a-d can be made from a variety of materials including, but not limited to, metal (e.g., tungsten, stainless steel, etc.) or a polymer. The lumen 410 can be a single lumen, as illustrated, or can alternatively comprise a plurality of independent lumens that each receive one or more of the drive cables 408a-d.

The drive cables 408a-d extend proximally from the end effector 204 to the drive housing 208 (FIG. 2) where they are operatively coupled to various actuation mechanisms or devices housed (contained) therein to facilitate longitudinal movement (translation) of the drive cables 408a-d within the lumen 410. Selective actuation of all or a portion of the drive cables 408a-d causes the end effector 204 (e.g., one or both of the jaws 210, 212) to articulate (pivot) relative to the shaft 202. More specifically, selective actuation causes a corresponding drive cable 408a-d to translate longitudinally within the lumen 410 and thereby cause pivoting movement of the end effector 204. One or more drive cables 408a-d, for example, may translate longitudinally to cause the end effector 204 to articulate (e.g., both of the jaws 210, 212 angled in a same direction), to cause the end effector 204 to open (e.g., one or both of the jaws 210, 212 move away from the other), or to cause the end effector 204 to close (e.g., one or both of the jaws 210, 212 move toward the other).

Moving the drive cables 408a-d can be accomplished in a variety of ways, such as by triggering an associated actuator or mechanism operatively coupled to or housed within the drive housing 208 (FIG. 2). Moving a given drive cable 408a-d constitutes applying tension (i.e., pull force) to the given drive cable 408a-d in a proximal direction, which causes the given drive cable 408a-d to translate and thereby cause the end effector 204 to move (articulate) relative to the shaft 202.

The wrist 206 includes a first plurality of pulleys 412a and a second plurality of pulleys 412b, each configured to interact with and redirect the drive cables 408a-d for engagement with the end effector 204. The first plurality of pulleys 412a is mounted to the proximal clevis 402b at the second axle 404b and the second plurality of pulleys 412b is also mounted to the proximal clevis 402b but at a third axle 404c located proximal to the second axle 404b. The first and second pluralities of pulleys 412a,b cooperatively redirect the drive cables 408a-d through an “S” shaped pathway before the drive cables 408a-d are operatively coupled to the end effector 204.

In at least one embodiment, one pair of drive cables 408a-d is operatively coupled to each jaw 210, 212 and configured to “antagonistically” operate the corresponding jaw 210, 212. In the illustrated embodiment, for example, the first and second drive cables 408a,b are coupled with a connector (not shown) at the first jaw 210, and the third and fourth drive cables 408c,d are coupled with a connector (not shown) at the second jaw 212. Consequently, actuation of the first drive cable 408a pivots the first jaw 210 about the first pivot axis P₁ toward the open position, and actuation of the second drive cable 408b pivots the first jaw 210 about the first pivot axis P₁ in the opposite direction and toward the closed position. Similarly, actuation of the third drive cable 408c pivots the second jaw 212 about the first pivot axis P₁ toward the open position, while actuation of the fourth drive cable 408d pivots the second jaw 212 about the first pivot axis P₁ in the opposite direction and toward the closed position.

Accordingly, the drive cables 408a-d may be characterized or otherwise referred to as “antagonistic” cables that cooperatively (yet antagonistically) operate to cause relative or tandem movement of the first and second jaws 210, 212. When the first drive cable 408a is actuated (moved), the second drive cable 408b naturally follows as coupled to the first drive cable 408a, and when the third drive cable 408c is actuated, the fourth drive cable 408d naturally follows as coupled to the third drive cable 408c, and vice versa.

The end effector 204 further includes a first jaw holder 414a and a second jaw holder 414b laterally offset from the first jaw holder 414a. The first jaw holder 414a is mounted to the first axle 404a and configured to receive and seat (e.g., “hold”) the first jaw 210 such that movement (rotation) of the first jaw holder 414a about the first pivot axis P₁ correspondingly moves (rotates) the first jaw 210. The first jaw holder 414a may also provide and otherwise define a first pulley 416a configured to receive and seat one or more drive cables, such as the first and second drive cables 408a,b to effect such movement (rotation). The second jaw holder 414b is similarly mounted to the first axle 404a and is configured to receive and seat (e.g., “hold”) the second jaw 212 such that movement (rotation) of the second jaw holder 414b about the first pivot axis P₁ correspondingly moves (rotates) the second jaw 212. The second jaw holder 414b may also provide and otherwise define a second pulley 416b configured to receive and seat one or more drive cables, such as the third and fourth drive cables 408c,d, to effect such movement (rotation).

The surgical tool 200 may also include an electrical conductor 418 that supplies electrical energy to the end effector 204, thereby converting the surgical tool 200 into an “electrosurgical instrument”. Similar to the drive cables 408a-d, the electrical conductor 418 may extend longitudinally within the lumen 410. In some embodiments, the electrical conductor 418 and the power cable 214 (FIG. 2) may comprise the same structure. In other embodiments, however, the electrical conductor 418 may be electrically coupled to the power cable 214, such as at the drive housing 208 (FIG. 2). In yet other embodiments, the electrical conductor 418 may extend to the drive housing 208 where it is electrically coupled to an internal power source, such as batteries or fuel cells.

In some embodiments, the electrical conductor 418 may comprise a wire that extends to one or both of the jaws 210, 212. In other embodiments, however, the electrical conductor 418 may comprise a rigid or semi-rigid shaft, rod, or strip (ribbon) made of a conductive material. In some embodiments, the electrical conductor 418 may be partially covered with an insulative covering made of a non-conductive material. The insulative covering, for example, may comprise a plastic applied to the electrical conductor 418 via heat shrinking, but could alternatively be any other non-conductive material.

In operation, the end effector 204 may be configured for monopolar or bipolar operation, without departing from the scope of the disclosure. Electrical energy is transmitted by the electrical conductor 418 to the end effector 204, which acts as an active (or source) electrode. In at least one embodiment, the electrical energy conducted through the electrical conductor 418 may comprise radio frequency (“RF”) energy exhibiting a frequency between about 100 kHz and 1 MHz. The RF energy causes ultrasonic agitation or friction, in effect resistive heating, thereby increasing the temperature of target tissue. Accordingly, electrical energy supplied to the end effector 204 is converted to heat and transferred to adjacent tissue to cut, cauterize, and/or coagulate the tissue (dependent upon the localized heating of the tissue), and thus may be particularly useful for sealing blood vessels or diffusing bleeding.

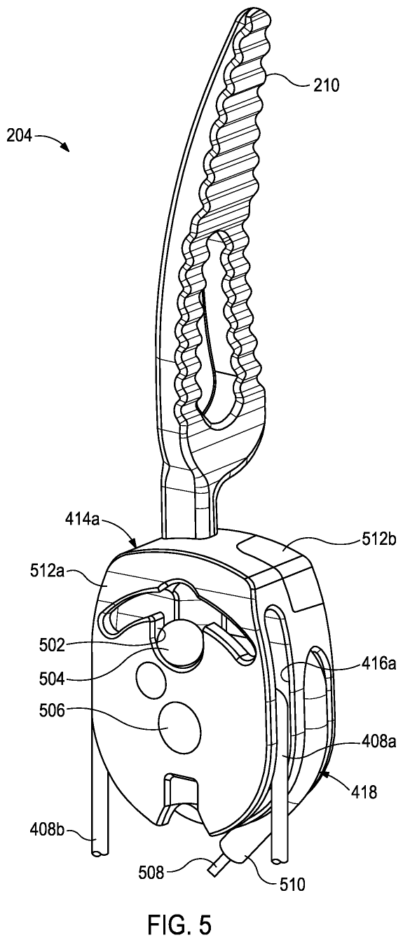

FIG. 5 is an enlarged isometric view of a portion of the end effector 204, according to one or more embodiments. More specifically, illustrated is the first jaw 210 mounted to the first jaw holder 414a. The following description, however, may be equally applicable to the second jaw 212 and the second jaw holder 414a. Moreover, while the first jaw 210 is shown, the principles of the present disclosure are equally applicable to other types of end effectors, such as a surgical hook. Accordingly, the first jaw 210 may be replaced with a surgical hook in some applications.

The jaw holder 414a secures the jaw 210 such that movement (rotation) of the jaw holder 414a during operation correspondingly moves (rotates) the jaw 210. As mentioned above, the jaw holder 414a provides and otherwise defines a pulley 416a configured to receive and seat one or more drive cables, such as the first and second drive cables 408a,b. A pocket 502 may be defined on the pulley 416a to receive and seat a connector 504, which couples the drive cables 408a,b to effect movement (rotation) of the jaw holder 414a as the drive cables 408a,b operate antagonistically. The jaw holder 414a also defines a central aperture 506 configured to receive the first axle 404a (FIG. 4) to rotatably mount the jaw holder 414a thereto.

A portion of the electrical conductor 418 is also depicted extending to the end effector 204 to provide electrical energy to the end effector 204 (i.e., the jaw 210), thereby converting the end effector 204 into an “electrosurgical instrument”. As illustrated, the electrical conductor 418 may include a supply conductor 508 encased in an insulation sheath 510. The supply conductor 508 may be electrically coupled to the jaw 210, thereby converting the jaw 210 into an active (or source) electrode for the end effector 204. In some applications, the end effector 204 could be configured for monopolar operation using only the electrical conductor 418. In other applications, however, the end effector 204 may be configured for bipolar operation. In such embodiments, a second electrical conductor may extend to and be electrically coupled to the second jaw 212 (FIGS. 2 and 4), and thereby making the second jaw 212 a return electrode.

The jaw holder 414a may be made of any electrically insulating or non-conductive material. Suitable non-conductive materials include, but are not limited to, a ceramic (e.g., zirconia, alumina, aluminum nitride, a silicate, silicon nitride, etc.), high temperature and high strength plastics, a thermoplastic or thermosetting polymer (e.g., polyether ether ketone or “PEEK”, ethylene tetrafluoroethylene or “ETFE”, polytetrafluoroethylene or “PTFE”, ULTEM™ VESPEL®, a polyphenylsulfone, a polysulfone, RADEL®, a polyamide-imide, a polyimide, an epoxy, etc.), a composite material (e.g., glass-filled, fiber-filled, etc.), a partially-filled composite material, hard rubber (e.g., ebonite), or any combination thereof.

As illustrated, the jaw holder 414a includes a first molded component 512a and a second molded component 512b coupled to the first molded component 512a. The first molded component 512a may be configured to receive and secure the jaw 210 and provide (define) the pulley 416a, and the second molded component 512b may be configured to fully encapsulate and insulate the supply conductor 508 at the jaw 210. The first molded component 512a may be molded (e.g., overmolded) onto the jaw 210 via a first injection molding shot, and the second molded component 512b may be overmolded onto the first molded component 512a via a second injection molding shot. In some cases, the first and second molded components 512a,b may be made of the same non-conductive material, but could alternatively be made of dissimilar non-conductive materials.

Since the molded components 512a,b of the jaw holder 414a are made of non-conductive materials and are formed to fully insulate the supply conductor 508 at the jaw 210, the electrical energy supplied to the jaw 210 may be effectively isolated from adjacent electrically-conductive parts of the end effector 204, such as the first axle 404a (FIG. 4) extending through the central aperture 506, the drive cables 408a,b, or the distal clevis 402a (FIG. 4) operatively coupled to the jaw holder 414a. Consequently, the electrical energy supplied to the jaw 210 may have a reduced risk of inadvertent tissue damage due to unintended current leakage to electrically-conductive parts of the end effector 204. Additionally, this method fully encapsulates the assembly, and reduces the number of parts, interfaces, and possible crevices where biomaterials may gather.

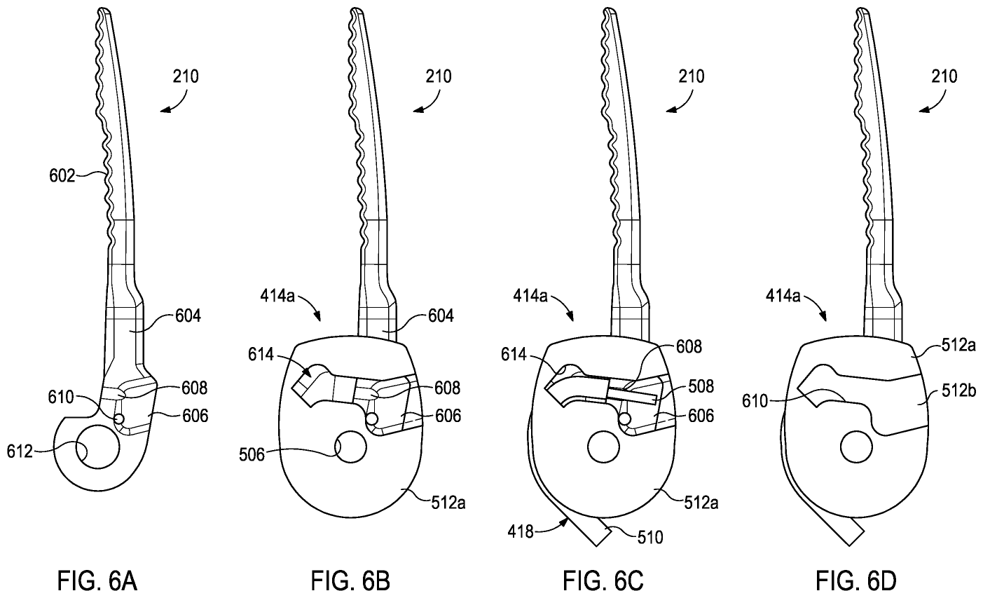

FIGS. 6A-6D illustrate progressive views of an example process of manufacturing the portion of the end effector 204 shown in FIG. 5. FIG. 6A is an isometric side view of the jaw 210, which may be made of a variety of electrically-conductive materials, such as a metal. In at least one application, the jaw 210 may be made of 17-4 stainless steel, but could alternatively be made of any conductive, high strength material that is resistant to chemical attack, such as a high strength stainless steel. In some applications, the jaw 210 may be coated to impart specific properties. For example, the jaw 210 may be coated to provide a release layer or to prevent corrosion. The jaw 210 may be machined from a larger piece of material or may alternatively be manufactured, such as through metal injection molding (MIM), machining, stamping, 3D printing, or any combination thereof.

As illustrated, the jaw 210 includes a tissue engagement portion 602, a shank 604 that extends from the tissue engagement portion 602, and a contact plate 606 coupled to or forming part of the shank 604. The contact plate 606 provides a location where the supply conductor 508 (FIG. 5) can be electrically coupled (e.g., welded) to the jaw 210 to convey electrical energy thereto. In some cases, the contact plate 606 may define a pinhole or “conductor channel” 608 through which the supply conductor 508 may extend to access and engage the contact plate 606.

As noted above, the principles of the present disclosure are equally applicable to other types of end effectors, such as surgical hooks. Similar to the jaw 210, a surgical hook may also include the shank 604 and the contact plate 606, but the tissue engagement portion 602 would be replaced with a hook-like feature or structure.

In some applications, the jaw 210 may also define a through hole 610 and an axle aperture 612. The through hole 610 may prove advantageous in helping couple the supply conductor 508 to the contact plate 606 and also help lock the jaw 210 to the jaw holder 414a (FIG. 5) via injection molding as the material of the injection molding flows therethrough. The axle aperture 612 may be configured to co-axially align with the central aperture 506 (FIG. 5) of the jaw holder 414a to receive the first axle 404a (FIG. 4) therethrough.

FIG. 6B is a front view of the jaw holder 414a following a first injection molding process resulting in the formation of the first molded component 512a. As illustrated, the first molded component 512a may be formed over a portion of the shank 604 and the contact plate 606. The first injection molding process may alternately be referred to as a first “overmold shot.” Moreover, the first molded component 512a defines the central aperture 506 of the jaw holder 414a, which aligns with the axle aperture 612 (FIG. 6A).

The first molded component 512a provides and otherwise defines a cable passage 614 that communicates with the contact plate 606 via the conductor channel 608. The electrical conductor 418 (FIG. 6C) may be routed through the cable passage 614 to allow the supply conductor 508 (FIG. 6C) to electrically communicate with (i.e., transmit electrical energy to) the contact plate 606.

FIG. 6C depicts the electrical conductor 418 received within the first molded component 512a of the jaw holder 414a. Following the first overmold shot that produces the first molded component 512a, the electrical conductor 418 may be routed through the cable passage 614 and placed in electrical communication with the jaw 210. A portion of the insulation sheath 510 may be stripped from the electrical conductor 418 to expose the supply conductor 508, and the exposed portion of the supply conductor 508 may be extended through the conductor channel 608 to access the contact plate 606.

The supply conductor 508 may then be coupled to the contact plate 606, such as through resistance welding or soldering. In some applications, resistance welding may be preferred over soldering as it provides a stronger bond without potentially hardening the electrical conductor 418 as a result of solder wicking.

FIG. 6D is a front view of the jaw 210 and the jaw holder 414a following a second injection molding process that forms the second molded component 512b. The second molded component 512b may be formed over the first molded component 512a and the injection pressure may be regulated to prevent excess flash. The second injection molding process may alternately be referred to as a second “overmold shot.” During the second overmold shot, the material for the second molded component 512b (e.g., plastic) fills and encapsulates a cavity 610 defined by the first molded component 512a. The cavity 610 may house or contain the contact plate 606 (FIG. 6C) and the exposed portion of the supply conductor 508 (FIG. 6C). Accordingly, the second overmold shot electrically isolates and insulates the contact plate 606 and the supply conductor 508. Moreover, the second overmold shot encapsulates and electrically isolates the supply conductor 508 and the jaw 210 from adjacent electrically-conductive parts of the end effector 204, which decreases the risk of inadvertent arcing due to creepage and clearance failure. The two overmolding shots also help fill in cracks and crevices in the end effector 204 that might present a bioburden risk.

The foregoing manufacturing steps for the jaw holder 414a and the jaw 210 may present various unique manufacturing challenges, typically associated with micro-molding very small parts, high grip and cable loads (high strength), electrical isolation, reusability, durability, and autoclavability (e.g., high temperatures). Such competing requirements might limit the number of available non-conductive materials (e.g., plastics, resins, etc.) that might be used for both the insulation sheath 510 and the jaw holder 414a. Moreover, the available material set and conflicting design requirements can potentially make maintaining an adequate process window very difficult. At one end of the spectrum, for example, fill pressures and temperatures might be too high and extreme flash could be generated at the conductor channel 608 (FIGS. 6A and 6B), alternately referred to as a “cable exit,” or the insulation sheath 510 might melt and reflow. At the other end of the spectrum, fill pressures and temperatures might be too low and interfacial adhesion between the first and second molded components 512a,b could be poor, or short shots might be generated. In some cases, both flash and short shots could be produced simultaneously, which might make it quite difficult to produce a defect-free part.

According to embodiments of the present disclosure, improved designs for the jaw holder 414a have been developed to provide a positive pressure shutoff on the electrical conductor 418 at the cable exit (i.e., the conductor channel 608) without risk of damage to the insulation sheath 510. Alternatively, or in addition thereto, improved designs for the jaw holder 414a have been developed to reduce or prevent the temperature and pressure of the advancing mold front at the cable exit. As described herein, the jaw holder is intended to enclose the insulation sheath in a manner such that the molten plastic does not compromise the insulation sheath when molten plastic is injected at elevated pressures. One way to accomplish this is to apply an amount of compression on the insulation sheath. Another way to accomplish this is to ensure that the jaw holder support is such that pressure drop across a longer supported region would oppose injection pressures.

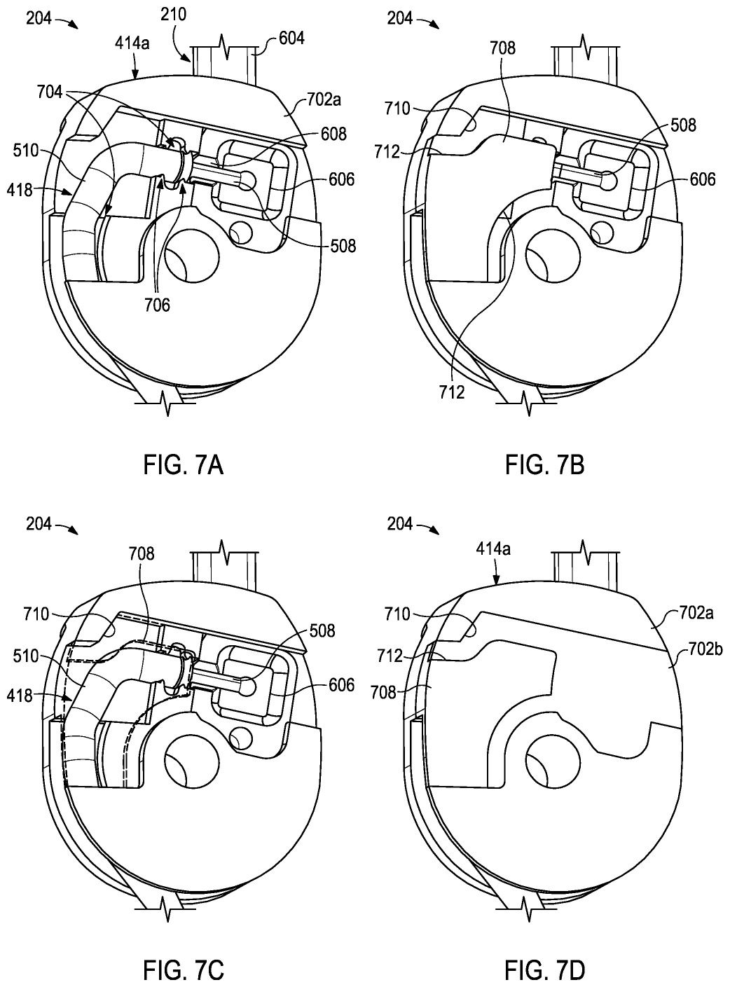

FIGS. 7A-7D illustrate progressive views of an example process of manufacturing the portion of the end effector 204 of FIG. 5, according to one or more embodiments of the present disclosure. The manufacturing process described in FIGS. 7A-7D may be similar in some respects to the manufacturing process shown and described with reference to FIGS. 6A-6D, and therefore may be best understood with reference thereto, where like numerals will represent like components not described again in detail.

In FIG. 7A, a first injection molding process has been completed to generate or provide a first molded component 702a that forms part of the jaw holder 414a. As illustrated, the first molded component 702a may be formed over (overmolded) a portion of the shank 604 and the contact plate 606 of the jaw 210.

The first molded component 702a provides and otherwise defines a cable passage 704 that communicates with the contact plate 606 via the conductor channel 608. The electrical conductor 418 may be routed through the cable passage 704 to allow the supply conductor 508 to electrically communicate with (i.e., transmit electrical energy to) the contact plate 606. A portion of the insulation sheath 510 may be stripped from the end of the electrical conductor 418 to expose the supply conductor 508, and the exposed portion of the supply conductor 508 may be extended through the conductor channel 608 to access the contact plate 606.

In some embodiments, one or more retention features 706 may be provided in the cable passage 704 at or near the conductor channel 608 to help capture the electrical conductor 418. More specifically, the retention features 706 may be configured to engage the insulation sheath 510 of the electrical conductor 418 and thereby help retain the electrical conductor 418 within the cable passage 704. As illustrated, the retention features 706 may comprise angled teeth or protrusions that extend radially into the cable passage 704, but could alternatively comprise any other type of structure capable of gripping the insulation sheath 510. In operation, the retention features 706 substantially prevent the electrical conductor 418 from reversing back through the cable passage 704. This may prove advantageous in providing strain relief on the electrical conductor 418 by helping to mitigate strain over several cycles of using the end effector 204. Moreover, the retention features 706 may prove advantageous in helping prevent the electrical conductor 418 from moving (shifting) during a second overmold shot, as discussed below.

In FIGS. 7B and 7C, a cable clip 708 is received within at least a portion of a cavity 710 defined in the first molded component 702a. The cable clip 708 is shown in FIG. 7C in phantom, thereby making the electrical conductor 418 visible. The cable clip 708 may comprise a component part that is molded and otherwise manufactured separate from the first molded component 702a. Moreover, the cable clip 708 may be sized and exhibit a geometry capable of being received within at least a portion of the cavity 710. In some embodiments, the cable clip 708 is received within the cavity 302 via an interference or shrink fit. In other embodiments, however, the cable clip 708 may be received within the cavity 710 via other suitable means, such as, for example, a press fit or one or more flexible mechanical fasteners.

The cable clip 708 may be configured to cover or entirely encapsulate the exposed portion of the insulation sheath 510 within the cavity 710. The interior of the cable clip 708 may be sized such that the electrical conductor 418 is compressed and firmly held in place when the cable clip 708 is received and secured within the cavity 710. In some embodiments, the cable clip 708 may be arranged within the cavity 710 prior to welding the supply conductor 508 to the contact plate 606 and thus may be configured to hold and properly locate the electrical conductor 418. Moreover, the cable clip 708 may provide positive pressure shutoff on the insulation sheath 510 prior to any subsequent injection molding process(es).

To assemble the jaw holder 414a, the first molded component 702a is first overmolded onto the shank 604 and portions of the contact plate 606. The electrical conductor 418 may then be mounted to the first molded component 702a, as generally described above, by routing the electrical conductor 418 through the cable passage 704 to allow the supply conductor 508 to electrically communicate with (engage) the contact plate 606. The cable clip 708 is then mounted to the first molded component 702a and otherwise received with the cavity 710 to cover or entirely encapsulate the exposed portion of the insulation sheath 510 within the cavity 710, as generally described above. After the cable clip 708 is received within the cavity 710, the supply conductor 508 may then be coupled to the contact plate 606, such as through resistance welding, laser welding, soldering, or the like. In other embodiments, however, it is contemplated herein that the supply conductor 508 may be welded to the contact plate 606 prior to installing the cable clip 708. In such embodiments, the cable clip 708 may form part of an assembly process configured to properly align the electrical conductor 418.

FIG. 7D is a front view of the end effector 204 following a second injection molding process that forms a second molded component 702b of the jaw holder 414a. The second molded component 702b may be formed over the first molded component 702a within the cavity 710 and at least a portion of the cable clip 708. The first and second molded components 702a,b and the cable clip 708 may be made of any of the nonconductive materials mentioned herein.

As illustrated, in some embodiments, the cable clip 708 may provide or define a raised shoulder 712 that extends around some or all of the cable clip 708. When the cable clip 708 is properly seated within the cavity 710, the raised shoulder 712 may be offset from the inner wall of the cavity 710 such that a gap is defined therebetween. During the second overmold shot, the material (e.g., plastic) for the second molded component 702b flows to fill and encapsulate the remainder of the cavity 710 and the gap, and otherwise the volume generated by the raised shoulder 712 over the cable clip 708. Accordingly, the second molded component 702b interlocks the first molded component 702a and the cable clip 708. Notably, inclusion of the cable clip 708 substantially or entirely prevents hot molten plastic from the second overmold shot from contacting the insulation sheath 510 of the electrical conductor 418, which could result in positive pressure shutoff of the insulation sheath 12. In this embodiment, tooling does not touch or damage the electrical conductor 418.

Benefits of the manufacturing process shown in FIGS. 7A-7D include, but are not limited to, a reduced risk of flash. Longer “serpentine” paths for the electrical conductor 418 are also achievable, and positive pressure on the electrical conductor 418 may be achieved with minimal risk of damage. Moreover, the cable clip 708 helps to divert the molten plastic from the second overmold shot away from the insulation sheath 510 of the electrical conductor 418. The cable clip 708 also serves as a workpiece holding assembly that aids in holding the electrical conductor 418 during the welding step and also during the second overmold shot.

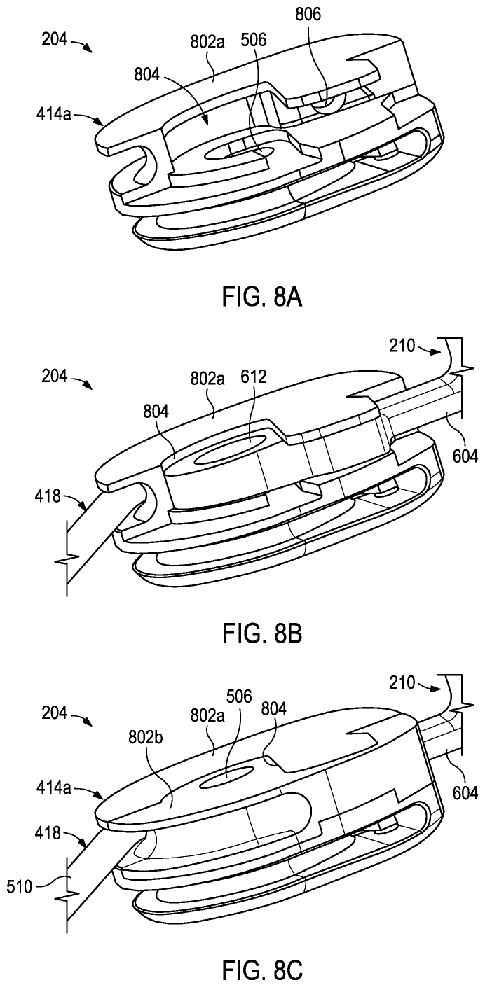

FIGS. 8A-8C illustrate progressive views of another example process of manufacturing the portion of the end effector 204 of FIG. 5, according to one or more additional embodiments of the present disclosure. The manufacturing process described in FIGS. 8A-8C may be similar in some respects to the manufacturing process shown and described with reference to FIGS. 6A-6D, and therefore may be best understood with reference thereto, where like numerals will again correspond to similar components not described again.

In FIG. 8A, a first injection molding process has been completed and thereby provides a first molded component 802a that forms part of the jaw holder 414a. As illustrated, the first molded component 802a may be formed separate from the jaw 210 (FIG. 8B) and otherwise not overmolded onto the jaw 210. Rather, the first molded component 802a may provide and otherwise define a cavity 804 configured to receive and seat the jaw 210 and, more particularly, at least a portion of the shank 604 (FIG. 8B) and the contact plate 606 (FIGS. 6A-6C) of the jaw 210.

The first molded component 802a defines a portion of the central aperture 506 of the jaw holder 414a, and may be configured to align with the axle aperture 612 (FIG. 8B) of the jaw 210. The first molded component 802a may also define a cable passage 806 that communicates with the cavity 804 and through which the electrical conductor 418 (FIG. 8B) may be extended to be electrically coupled to the jaw 210.

In FIG. 8B, the jaw 210 and the electrical conductor 418 are shown mounted to the first molded component 802a and, more particularly, received within the cavity 804. As illustrated, the axle aperture 612 of the jaw 210 is aligned with the portion of the central aperture 506 (FIG. 8A) defined by the first molded component 802a. In some embodiments, the electrical conductor 418 and, more specifically, the supply conductor 508 (FIGS. 5B and 5C) may be electrically coupled (welded) to the jaw 210 prior to installation on the first molded component 802a. In such embodiments, the electrical conductor 418 may be threaded through the cable passage 408 (FIG. 8A) until the jaw 210 is able to be properly received and seated within the cavity 804. Moreover, in such embodiments, an adhesive may be applied to the electrical conductor 418 at or near the exit to the cable passage 806. The adhesive may help with electrical insulation while simultaneously sealing the interface at the exit for the cable passage 806, which could prevent entrapment of bioburden, etc. In other embodiments, however, the supply conductor 508 may be electrically coupled to the jaw 210 after the jaw 210 is received within the cavity 804 of the first molded component 802a.

In FIG. 8C, a second molded component 802b is provided on the jaw holder 414a following a second injection molding process. The second molded component 802b may be overmolded onto the first molded component 802a and the exposed portions of the jaw 210 and the shank 604 situated within the cavity 804. During the second overmold shot, the material for the second molded component 802b (e.g., plastic) fills and encapsulates the remainder of the cavity 804, and the second molded component 802b further defines the remaining portion of the central aperture 506 of the jaw holder 414a.

Notably, the high-pressure, high-temperature material of the second molded component 802b may not contact the insulation sheath 510 of the electrical conductor 418. In particular, in the foregoing manufacturing process, the electrical conductor 418 is loaded into the first molded component 802a and tooling does not touch or damage the electrical conductor 418. Advantageously, this avoids the pressure of the hot material from contacting the insulation sheath 510 of the electrical conductor 418.

Benefits of the manufacturing process shown in FIGS. 8A-8C include, but are not limited to, improved shutoff around the electrical conductor 418 during overmolding. In particular, the shut off surfaces of the second molded component 802b are separated from the electrical conductor 418. Moreover, the critical bend area of the electrical conductor 418 may be at a lower risk of experiencing flash. Furthermore, there may be improved electrical isolation around the inside face of the jaw 210, and welding of the electrical conductor 418 and the jaw shank 604 may be performed independently of injection molding, which may lower risk of cable damage.

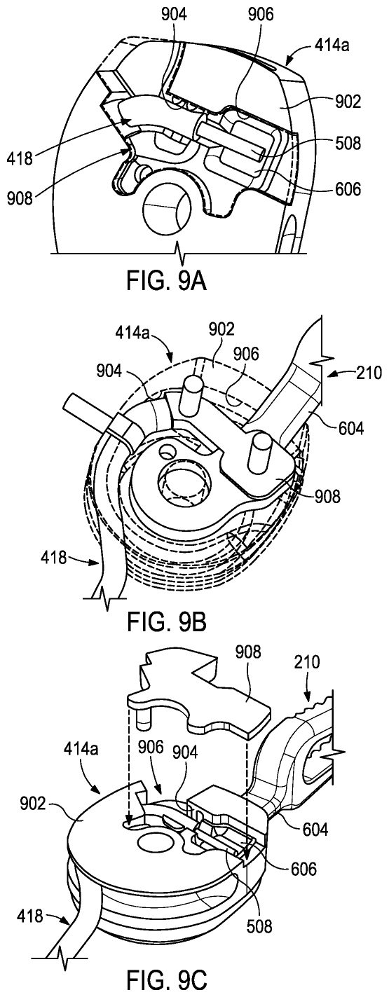

FIGS. 9A-9C depict alternate examples of manufacturing the jaw holder 414a of the end effector 204 of FIG. 5, according to one or more additional embodiments. The manufacturing processes described in FIGS. 9A-9C may be similar in some respects to the manufacturing process shown and described with reference to FIGS. 6A-6D, and therefore may be best understood with reference thereto, where like numerals will again correspond to like components not described again.

Each of FIGS. 9A-9C depicts a different way to manufacture the jaw holder 414a, but each embodiment may be similar in some respects. In each embodiment of FIGS. 9A-9C, for instance, a first injection molding process is undertaken to generate or produce a molded component 902 that forms part of the jaw holder 414a. As illustrated, the molded component 902 is formed over the jaw 210 (only visible in FIGS. 9B and 9C) and, more particularly, over a portion of the shank 604 and the contact plate 606 (only visible in FIGS. 9A and 9C). The molded component 902 provides and otherwise defines a cable passage 904 configured to receive the electrical conductor 418, which allows the supply conductor 508 (only visible in FIGS. 9A and 9C) to electrically communicate with (i.e., transmit electrical energy to) the contact plate 606.

As illustrated, the molded component 902 may define a cavity 906 which exposes portions of the jaw 210 and the electrical conductor 418 and, more particularly, the location where the supply conductor 508 is electrically coupled to the contact plate 606. In each of the embodiments shown in FIGS. 9A-9C, a second overmold shot, as described in previous embodiments, is eliminated, thus eliminating the high-pressure, high-temperature injection molding process that can potentially damage the electrical conductor 418. Instead, in the embodiments of FIGS. 9A-9C, the cavity 906 may be filled or covered with a low-temperature and low-pressure filler material 908 that is not overmolded at high-temperature or high-pressure. The molded component 902 carries the bulk of the mechanical load for the jaw holder 414a, and the filler material 908 is subsequently applied to the molded component 902.

In FIG. 9A, the filler material 908 comprises a potting of an epoxy material received within and filling the cavity 906. In such embodiments, the epoxy material may be flowed into the cavity 906 at low-pressure (e.g., ambient pressure) and low-temperature (e.g., ambient temperature) and allowed to set, thereby fully encapsulating and insulating the supply conductor 508 at the jaw 210.

In FIG. 9B, the filler material 908 comprises a low-temperature and low-pressure casting or mold received within and filling the cavity 906. In such embodiments, the geometry and shape of the casting or mold for the filler material 908 will be configured to mate with the cavity 906. The filler material 908 may be received and secured within the cavity 906 via a variety of coupling means including, but not limited to, low pressure injection molding, low pressure compression molding, an interference or shrink fit, a press fit, one or more mechanical fasteners, an adhesive, or any combination thereof. In at least one embodiment, the material for the casting or mold may comprise silicone. The pins visible in FIG. 9B and extending from the casting are molded gates where the material is fed into the cavity 504. The pins would be removed subsequent to the molding process.

In FIG. 9C, the filler material 908 comprises a molded or formed cover that exhibits a geometry configured to be received within the cavity 504. The cover may be made of any of the non-conductive materials mentioned herein. The cover may be secured to the molded component 902 via a variety of coupling means including, but not limited to, an interference or shrink fit, a press fit, one or more mechanical fasteners, an adhesive, or any combination thereof. In embodiments where an adhesive is used, the adhesive may help provide electrical isolation.

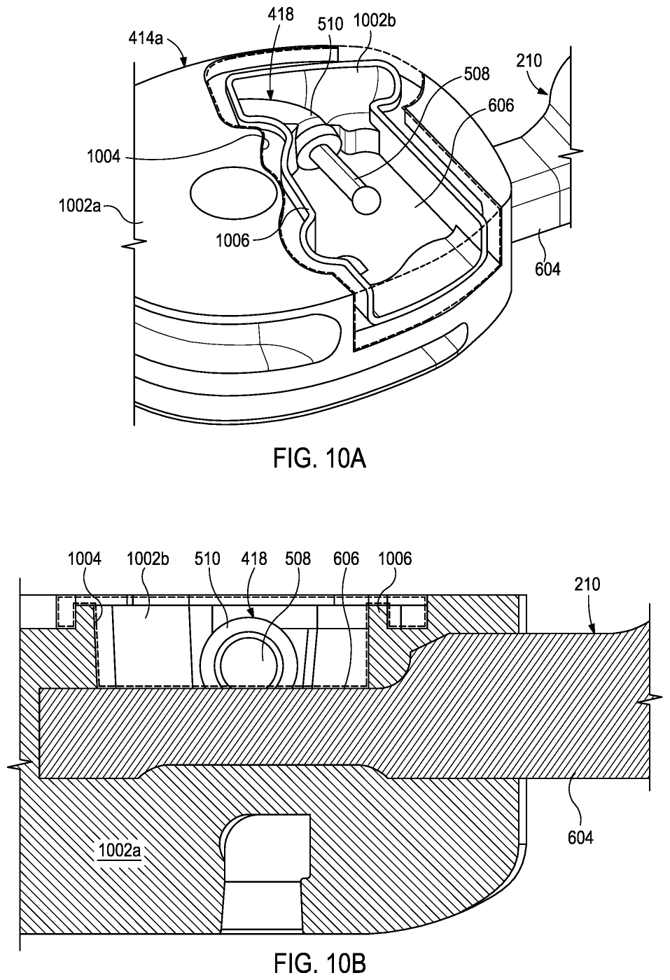

FIGS. 10A and 10B are isometric and cross-sectional side views of another example method of manufacturing the jaw holder 414a of the end effector 204 of FIG. 5, according to one or more additional embodiments. The method of manufacturing the jaw holder 414a shown in FIGS. 10A-10B may be similar in some respects to the manufacturing method shown in FIGS. 6A-6D herein, and therefore may be best understood with reference thereto, where like numerals will correspond to like compliments not described again in detail.

As illustrated, the jaw holder 414a may be manufactured by first producing a first molded component 1002a overmolded onto a portion of the shank 604 and the contact plate 606 (FIG. 10A) of the jaw 210. The first molded component 1002a may define a cavity 1004. The electrical conductor 418 may then be mounted to the first molded component 1002a, as generally described herein, and the supply conductor 508 may be electrically coupled (e.g., welded) to the contact plate 606 to electrically communicate with the jaw 210.

A second injection molding process may then be undertaken to produce a second molded component 1002b that forms the rest of the jaw holder 414a. The second molded component 1002b may be formed over the first molded component 1002a and configured to fill the cavity 1004 and thereby encapsulate the exposed portions of the electrical conductor 418, the supply conductor 508, and the contact plate 606.

In prior two-shot jaw holders, the interfacial adhesion between the first and second shots could be poor and lead to loss of electrical isolation following mechanical cycling and cleaning and sterilization. To achieve an adequate interfacial bond between the first and second shots, while maintaining electrical isolation throughout the life of the device, the material of the first shot preferably melts and mixes with the material of the second shot as the cavity fills.

In the present embodiment, to ensure that an adequate amount of heat is transferred to the first molded component 1002a and that shear mixing occurs before the second molded component 1002b solidifies in the cavity 1004, the first molded component 1002a may include and otherwise define a raised thin-walled rib 1006 that facilitates a lap joint with the second molded component 1002b. In at least one embodiment, as illustrated, the raised thin-walled rib 1006 may circumscribe the interior of the cavity 1004 and otherwise follow the same geometry as the cavity 1004.

As the molten material of the second molded component 1002b fills the cavity 1004, the hot mold front of the material of the second shot is compressed in the thinner, lap joint region facilitated by the thin-walled rib 1006 and intermixing flow velocities increase. Shear mixing of the materials of the first and second shots at higher velocities raises the local temperature of the mold front in the raised thin-walled rib 1006. The combination of higher temperatures and shear mixing melts the raised thin-walled rib 1006 and creates an adequate bond. This may be preferred over increasing the mold temperature, since increasing the mold temperature could result in adverse consequences, such as degradation of material in the barrel if the process must be paused for any reason, increased risk of melting of the insulation sheath 510 of the electrical conductor 418, increased risk of flash around the electrical conductor 418, and others.

The embodiment shown in FIGS. 10A-10B helps to eliminate interfacial adhesion risk between the first and second shots; i.e., between the first and second molded components 602a,b. The resulting bond between the first and second molded components 602a,b has improved electrical safety in eliminating or mitigating common electrical leakage paths. Mechanical robustness of the jaw holder 414a has also improved and shown reduced separation/cracking under load during testing.

Embodiments disclosed herein include:

A. An end effector for a surgical tool includes a jaw including a shank and a contact plate forming part of the shank, and a jaw holder secured to the jaw and providing a first molded component defining a cavity and a cable passage that communicates with the contact plate, and a cable clip received within the cavity. The end effector further includes a second molded component overmolded onto the first molded component and at least a portion of the cable clip, and filling the cavity, and an electrical conductor extending through the cable passage and including an insulation sheath and a supply conductor electrically coupled to the contact plate to supply electrical energy to the jaw, wherein the cable clip covers an exposed portion of the insulation sheath within the cavity, and wherein the second molded component encapsulates and electrically insulates the contact plate and an exposed portion of the supply conductor.

B. An end effector for a surgical tool includes a jaw including a shank and a contact plate forming part of the shank, a jaw holder secured to the jaw and providing a first molded component defining a cavity and a cable passage that communicates with the contact plate, and a second molded component overmolded onto the first molded component and filling the cavity. The end effector further including an electrical conductor extending through the cable passage and including an insulation sheath and a supply conductor electrically coupled to the contact plate to supply electrical energy to the jaw, wherein the second molded component encapsulates and electrically insulates exposed portions of the jaw and the shank situated in the cavity without contacting the insulation sheath.

C. An end effector for a surgical tool includes a jaw including a shank and a contact plate forming part of the shank, a jaw holder secured to the jaw and providing a molded component overmolded onto portions of the shank and the contact plate and defining a cable passage that communicates with the contact plate, an electrical conductor extending through the cable passage and including an insulation sheath and a supply conductor electrically coupled to the contact plate to supply electrical energy to the jaw, a cavity defined in the jaw holder and exposing portions of the jaw and the electrical conductor, and a filler material that covers exposed portions of the jaw and the electrical conductor within the cavity.

D. An end effector for a surgical tool includes a jaw including a shank and a contact plate forming part of the shank, a jaw holder secured to the jaw and providing a first molded component defining a cavity, a cable passage that communicates with the contact plate, and a raised thin-walled rib that circumscribes at least a portion of the interior of the cavity, and a second molded component overmolded onto the first molded component and filling the cavity. The end effector further including an electrical conductor extending through the cable passage and including a supply conductor electrically coupled to the contact plate to supply electrical energy to the jaw, wherein the second molded component encapsulates and electrically insulates the contact plate and an exposed portion of the supply conductor, and wherein molten material of the second molded component melts and intermixes with the material of the raised thin-walled rib.

Each of embodiments A, B, C, and D may have one or more of the following additional elements in any combination: Element 1: further comprising one or more retention features defined in the cable passage and arranged to engage and retain the electrical conductor within the cable passage. Element 2: wherein the first molded component is overmolded on top of a portion of the shank and the contact plate. Element 3: wherein the cable clip is received within the cavity via at least one of an interference fit, a shrink fit, a press fit, or one or more flexible mechanical fasteners, and any combination thereof. Element 4: wherein the cable clip defines a raised shoulder and material of the second molded component fills a volume generated by the raised shoulder over the cable clip. Element 5: wherein the first and second molded components and the cable clip are made of a non-conductive material selected from the group consisting of a plastic, a thermoplastic or thermosetting polymer, a composite material, and any combination thereof.

Element 6: wherein the jaw defines an axle aperture and the first and second molded components cooperatively define a central aperture of the jaw holder to align with the axle aperture. Element 7: wherein the first molded component is overmolded on top of a portion of the shank and the contact plate. Element 8: wherein the first molded component is formed separate from the jaw and the jaw is subsequently mounted to the first molded component. Element 9: wherein the electrical conductor is threaded through the cable passage until the jaw is received and seated within the cavity. Element 10: further comprising an adhesive applied at an interface between the cable passage and the electrical conductor to prevent entrapment of bioburden within the cable passage.

Element 11: wherein the filler material comprises a potting of an epoxy material received within and filling the cavity. Element 12: wherein the filler material comprises a low-temperature and low-pressure casting received within and filling the cavity. Element 13: wherein the low-temperature and low-pressure casting comprises silicone. Element 14: wherein the filler material comprises a cover that exhibits a geometry configured to be received within the cavity. Element 15: wherein the cover is made of a non-conductive material and is secured to the molded component using an adhesive.

Element 16: wherein the electrical conductor includes an insulation sheath, and the molten material of the second molded component contacts the insulation sheath.

By way of non-limiting example, exemplary combinations applicable to A, B, C, and D include: Element 8 with Element 9; Element 12 with Element 13; and Element 14 with Element 15.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is:

1. An end effector for a surgical tool, comprising:
a jaw including a shank and a contact plate forming part of the shank;
a jaw holder secured to the jaw and providing:
a first molded component defining a cavity and a cable passage that communicates with the contact plate;
a cable clip received within the cavity; and
a second molded component overmolded onto the first molded component and at least a portion of the cable clip, and filling the cavity; and
an electrical conductor extending through the cable passage and including an insulation sheath and a supply conductor electrically coupled to the contact plate to supply electrical energy to the jaw,
wherein the cable clip covers an exposed portion of the insulation sheath within the cavity, and
wherein the second molded component encapsulates and electrically insulates the contact plate and an exposed portion of the supply conductor.

2. The end effector of claim 1, further comprising one or more retention features defined in the cable passage and arranged to engage and retain the electrical conductor within the cable passage.

3. The end effector of claim 1, wherein the first molded component is overmolded on top of a portion of the shank and the contact plate.

4. The end effector of claim 1, wherein the cable clip is received within the cavity via at least one of an interference fit, a shrink fit, a press fit, or one or more flexible mechanical fasteners, and any combination thereof.

5. The end effector of claim 1, wherein the cable clip defines a raised shoulder and material of the second molded component fills a volume generated by the raised shoulder over the cable clip.

6. The end effector of claim 1, wherein the first and second molded components and the cable clip are made of a non-conductive material selected from the group consisting of a plastic, a thermoplastic or thermosetting polymer, a composite material, and any combination thereof.

7. An end effector for a surgical tool, comprising:
a jaw including a shank and a contact plate forming part of the shank;
a jaw holder secured to the jaw and providing:
a first molded component defining a cavity and a cable passage that communicates with the contact plate; and
a second molded component overmolded onto the first molded component and filling the cavity; and
an electrical conductor extending through the cable passage and including an insulation sheath and a supply conductor electrically coupled to the contact plate to supply electrical energy to the jaw,
wherein the second molded component encapsulates and electrically insulates exposed portions of the jaw and the shank situated in the cavity without contacting the insulation sheath.

8. The end effector of claim 7, wherein the jaw defines an axle aperture and the first and second molded components cooperatively define a central aperture of the jaw holder to align with the axle aperture.

9. The end effector of claim 7, wherein the first molded component is overmolded on top of a portion of the shank and the contact plate.

10. The end effector of claim 7, wherein the first molded component is formed separate from the jaw and the jaw is subsequently mounted to the first molded component.

11. The end effector of claim 10, wherein the electrical conductor is threaded through the cable passage until the jaw is received and seated within the cavity.

12. The end effector of claim 7, further comprising an adhesive applied at an interface between the cable passage and the electrical conductor to prevent entrapment of bioburden within the cable passage.

13. An end effector for a surgical tool, comprising:
a jaw including a shank and a contact plate forming part of the shank;
a jaw holder secured to the jaw and providing a molded component overmolded onto portions of the shank and the contact plate and defining a cable passage that communicates with the contact plate;
an electrical conductor extending through the cable passage and including an insulation sheath and a supply conductor electrically coupled to the contact plate to supply electrical energy to the jaw;
a cavity defined in the jaw holder and exposing portions of the jaw and the electrical conductor; and
a filler material that covers exposed portions of the jaw and the electrical conductor within the cavity.

14. The end effector of claim 13, wherein the filler material comprises a potting of an epoxy material received within and filling the cavity.

15. The end effector of claim 13, wherein the filler material comprises a low-temperature and low-pressure casting received within and filling the cavity.

16. The end effector of claim 15, wherein the low-temperature and low-pressure casting comprises silicone.

17. The end effector of claim 13, wherein the filler material comprises a cover that exhibits a geometry configured to be received within the cavity.

18. The end effector of claim 17, wherein the cover is made of a non-conductive material and is secured to the molded component using an adhesive.

19. An end effector for a surgical tool, comprising:
a jaw including a shank and a contact plate forming part of the shank;
a jaw holder secured to the jaw and providing:
a first molded component defining a cavity, a cable passage that communicates with the contact plate, and a raised thin-walled rib that circumscribes at least a portion of the interior of the cavity; and
a second molded component overmolded onto the first molded component and filling the cavity; and
an electrical conductor extending through the cable passage and including a supply conductor electrically coupled to the contact plate to supply electrical energy to the jaw,
wherein the second molded component encapsulates and electrically insulates the contact plate and an exposed portion of the supply conductor, and
wherein molten material of the second molded component melts and intermixes with the material of the raised thin-walled rib.

20. The end effector of claim 19, wherein the electrical conductor includes an insulation sheath, and the molten material of the second molded component contacts the insulation sheath.
